(19) United States
(12) Patent Application Publication
(10) Pub. No.: US 2003/0175362 A1
(43) Pub. Date: Sep. 18, 2003

(54) Disinfecting nitrous acid compositions and process for using the same

(76) Inventors: Robert D. Kross, Bellmore, NY (US); Lorrence H. Green, Westbury, NY (US)

Correspondence Address: H.D. Coleman, Coleman Sudol Sapone, P.C., 714 Colorado Avenue, Bridgeport, CT, 06605-1601, US

(21) Appl. No.: 10/041,310

(22) Filed: Jan. 7, 2002

(51) Int. Cl. A61K 33/00

(52) U.S. Cl. 424/718

(57) ABSTRACT

The present invention relates generally to compositions and methods for the use of nitrous acid solutions to disinfect inanimate surfaces and animal tissues, and to treat diseases and wounds. More specifically, the invention deals with the partial and selected conversion of nitrite ion to nitrous acid in order to optimize the germicidal efficacy and duration of the nitrous acid consistent with the nature of the intended application.

### FIELD OF THE INVENTION

This invention relates generally to compositions and methods for the use of nitrous acid solutions to disinfect inanimate surfaces and animal tissues, and to treat diseases and wounds. More specifically the invention deals with the partial and selected conversion of nitrite ion to nitrous acid in order to optimize the germicidal efficacy and duration of the nitrous acid consistent with the nature of the intended application.

### BACKGROUND OF THE INVENTION

Within the last 20 years, a powerful disinfecting technology has emerged which relies on the combination of an inactive oxyanion and a suitable proton donor, such combination of elements occurring shortly before the product's intended use. The subsequent degradation of the resulting acid into a series of transient, cidal oxidants provides a heretofore unparalleled means for killing or inactivating a broad spectrum of bacteria, yeasts, molds and viruses in a very rapid manner, and in high numbers. Specifically this description refers to the in-situ creation of metastable chlorous acid in aqueous chlorite (ClO₂⁻) solutions, under conditions where the chlorous acid, HClO₂, represents a relatively small fraction of the total chlorite ion present, typically no more than about 15%, in order to minimize the otherwise rapid degradation of the system. The antimicrobially-effective chlorous acid systems function at pH values from about 3.5 down to about 2.6.

The protic acid source to effect this conversion is generally an organic acid (U.S. Pat. Nos. 4,986,990, 5,185,161), although inorganic acids (U.S. Pat. No. RE 36,064) and even acid-inducing metal salts have been taught (U.S. Pat. No. 5,820,822), in the extended series of patents which disclose the various aspects of this technology. The acidified chlorite compositions were first taught by Alliger in 1978 (U.S. Pat. No. 4,084,747) and in 1982 (U.S. Pat. No. 4,330,531), where the acid activator was lactic acid, which was deemed critical to the unique activity of the acid/chlorite system.

Subsequent prior art taught the creation of a diverse range of acidified chlorite compositions and their method of use. These included U.S. Pat. Nos. 4,891,216 (for topical application); 4,956,184 (for genital herpes); 5,100,652 (for oral hygiene); 5,384,134 (for anti-inflammatory activity); 5,389,390 and 6,063,425 (for disinfecting poultry and other meats); 5,597,561 and 5,651,977(adherent topical disinfectants); 5,628,959 (sterilizing hemodialyzers); 5,772,985 (bovine warts); 6,096,350 (for honey bee diseases); and 6,123,966 (stabilized disinfecting compositions).

In seven of these disclosures, one of the present inventors (Kross) was cited as the sole inventor, and was one of two or three cited inventors in all the remaining patent disclosures. And as such, he became very familiar with the capabilities and deficiencies of the acid/oxy anion system, based upon chlorite. Although the capabilities of the acidified chlorite system are extensive, several inherent characteristics are present which limit its application in certain situations. The major difficulty lies in the relatively strong oxidizing tendency of the system, and the particularly corrosive effects of the chlorine dioxide (ClO₂), which forms upon degradation of the chlorous acid. ClO₂ will corrode many of the metals used in the fabrication of medical and dental equipment, as well as the metals associated with equipment used to dispense the solutions for such applications as the commercial disinfection of poultry, meats and agricultural commodities. A further detriment of the acidified chlorite systems is the noxiousness of the ClO₂ gas, for which OSHA has listed a very low permissible concentration in the air to which workers may be exposed for an 8 hour period. That level, 0.1 parts per million in the air, is 10 times lower, for example, than for chlorine, for which OSHA has listed a maximum permissible level of 1.0 ppm over an 8-hour period.

In researching the seeming uniqueness of the uninegative chlorite ion, it became evident that there is another oxyanion, namely the nitrite ion, that is strikingly similar to that of chlorite (see, for example, Friedman's “On the Ultraviolet Absorption Spectra of Uninegative Ions,” re: the electronic properties of both ions). Both form unstable acid counterparts, Le. chlorous and nitrous acids, with increasing instability as the acid form represents a greater and greater fraction of the acidified oxyanion solution. Neither acid can be isolated. Nitrogen appears in at least 8 oxidation states in its water soluble species; chlorine has at least 6. In general, species such as nitrous and chlorous acids, which have intermediate oxidation numbers, will be unstable with respect to disproportionation. The degradation of both these acids leads to the formation of gases (chlorine dioxide and nitric oxide [NO]) which are unique in possessing unpaired electrons. Both of these materials have unusual properties. Chlorine dioxide has become an excellent replacement for chlorine in water disinfection, by virtue of its high biocidal activity without formation of chloro-organic mutagens. It has also found use in the disinfection of food. In both these cases the chlorine dioxide degrades through several steps, through a 5-electron transfer, to innocuous chloride ion. With respect to nitric oxide, while it is one of the simplest biological molecules in nature, it has recently found its way into nearly every phase of biology and medicine. This ranges from its role as a critical endogenous regulator of blood flow and thrombosis, to a principal neurotransmitter mediating erectile function, to a major pathophysiological mediator of inflammation and host defense. These major discoveries have stimulated intense and extensive research into a vast array of fields including chemistry, molecular biology, and gene therapy. And consistent with the many functions of NO in the body, is the much greater allowable air levels of NO in workplace air, as listed by OSHA. For an 8-hour continuous exposure, that agency allows up to 25 ppm of NO in the air, as cf. the 0.1 ppm allowed for chlorine dioxide under similar exposure conditions.

Nitric oxide is most commonly produced by degradation of nitrites, long used for the curing of meat. And, with respect to the safety of nitrites, several recent scientific developments have affirmed the safety of nitrite as a curing agent, and its essential role in protecting the public health, as reported to a panel of food safety experts at the 2001 Annual Meeting of the Institute of Food Technologists. For example, the National Toxicology Program completed a study in 2000 which produced no meaningful evidence that nitrites cause cancer in rats and mice, an otherwise long-held suspicion. In fact, nitrite actually showed very strong protective effects against cancer and reduced tumor incidence in rodents fed the highest doses. Further evidence indicated nitrite's statistically-significant ability to prevent leukemia, which some epidemiologists had suggested was associated with sodium nitrite.

One difference between the two paramagnetic, unpaired-electron chlorine dioxide and nitric oxide molecules, which derive respectively from the degradation of chlorous acid and nitrous acid solutions, is that the chlorine in chlorine dioxide has lost one electron with respect to that in chlorous acid (i.e. a +4 charge in the former vs. +3 in the latter), whereas the nitrogen in nitric oxide, with a +2 charge, has gained an electron as cf. the +3 charge of the nitrite nitrogen. This difference may lead to an advantage in the potential metal corrosivity of chlorous acid/chlorine dioxide and nitrous acid/nitric oxide systems. In the latter regard, sodium nitrite per se has been employed in the corrosive inhibition of metals. Nitrous acid/nitrite solutions, if they possess appropriate antimicrobial activity, therefore, may have value in the disinfection of metallic articles, where acidified chlorite systems cannot readily be employed.

Nitrite has a long history of use in curing meat and preserving the red coloration of meat. It is well-established that addition of sodium nitrite to processed meat products prevents the growth of, and the toxin formation by Clostridium botulinum. Nitrite has also been reported to have inhibitory effects against other important food-borne microorganisms, such as Listeria monocytogenes, Escherichia coli, Enterobacter, Flavobacterium, Micrococcus and Pseudomonas. Nitric oxide per se has also found use as a curing agent. In U.S. Pat. No. 6,099,881, Hanson showed that exposure of meat to NO leads to its reaction with moisture in the food product to produce nitrous acid, which diffuses through the product, to produce the pink “cured” color pigment. In the curing reaction, NO reacts with myoglobin to give a red pigment. And, as an interesting insight regarding the antimicrobial properties of NO, a researcher at the St. Bartholomew's and Royal London School of Medicine and Dentistry has validated the age-old practice of licking wounds to clean and disinfect them. Saliva contains significant levels of nitrite, which can be converted to nitric oxide by salivary enzymes. The researcher had 14 healthy subjects lick all over their hands and then he measured the synthesis of nitric oxide on their skin. Nitric oxide levels increased sharply, suggesting that nitric oxide derived from salivary nitrite applied to the skin contributes to the antimicrobial effects of wound licking.

It appeared appropriate therefore, by virtue of 1)- the similar chemistry of the acidified nitrite as cf the chlorite system, 2)- its possible role in producing antimicrobial activity paralleling that of acidified chlorite, 3)- a possible advantage in reduced corrosion and animal tissue decolorization and 4)- a definite advantage with respect to inhalation toxicity of its NO breakdown product, to investigate, understand, and optimize the acidified nitrite system as an antimicrobial agent. Activity would be evaluated as a function of the nitrous acid/nitrite ratios in test solutions, as controlled by available hydrogen ion, and with particular regard to the potential use of acidified nitrite in disinfecting medical and related instrumentation; materials which are adversely affected by acidified chlorite disinfecting systems Accordingly, this invention is a result of the search for a controllable antimicrobial system to parallel the excellence of the acidified chlorite system while eliminating the corrosive aspects, tissue decolorization, and health impact of that technology.

### OBJECTS OF THE INVENTION

It is, therefore, an object of the present invention to provide antimicrobial acidified nitrite solutions.

It is a further object of the invention to control the antimicrobial action of these solutions by modifying their acidity and thus the degree of conversion of nitrite ion to nitrous acid, the presumptive source of germicidal action.

It is yet a further object of the invention to provide compositions based upon nitrous acid which exhibit rapidity and spectrum of action against representative species of the various microbial types.

It is still another object of the invention to provide acidified nitrite solutions which exhibit significant antimicrobial activity with minimum corrosion of medical, dental and other metallic substrates which require disinfection.

It is an additional object of the invention to provide acidified nitrite solutions which exhibit antimicrobial activity with minimum decolorization and bleaching of animal tissue substrates.

These, and/or other objects of the present invention will become apparent from a review of the following summary of the invention and description of the preferred embodiments.

### SUMMARY OF THE INVENTION

In one aspect, the present invention provides a composition for disinfecting a substrate using a nitrous acid generating composition. This composition comprises an aqueous solution containing a suitable amount of a protic acid, or a material inducing an acidic environment therein, and a suitable amount of a metal nitrite. The nitrite ion concentration in the form of nitrous acid is no more than about 95% by weight of the total amount of nitrite ion concentration.

In a preferred embodiment of this aspect of the present invention, there is provided a composition for disinfecting a substrate with a composition comprising a nitrous acid generating compound with a sufficient amount of a suitable organic acid to lower the pH of the composition to less than about 7. The preferred organic acid is an alpha hydroxy acid which has the formula:

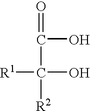

R¹ and R² may be the same or different and may be selected from the group consisting of hydrogen, methyl, —CH₂COOH, —CH₂COO⁻, —CH₂OH, —CHOHCOOH, —C₆H₅, and —CH₂C₆H₅ The pKₐ of the organic acid may be from about 2.8 to about 4.8.

In another preferred embodiment of this aspect of the present invention, there is provided a composition for disinfecting a substrate with a composition comprising a nitrous acid generating compound with an amount of phosphoric acid (pKₐ=2.15) sufficient to lower the pH of the composition to less than about 7.

In another aspect, the present invention provides processes for disinfecting a substrate using the compositions described above. These processes comprise applying the compositions described above to a substrate in order to disinfect the substrate.

In yet another aspect, the present invention provides a process for preparing these disinfecting compositions and separately, for disinfecting a surface using the resulting nitrous acid containing composition. The process comprises contacting the protic acid, or a solution with induced acidity, with the metal nitrite to form the disinfecting compositions, which are used in effective amounts to disinfect a desired surface.

### DETAILED DESCRIPTION OF THE INVENTION AND PREFERRED EMBODIMENTS

In describing the present invention, the following terms will be used.

The term “nitrite” or “nitrite salt” is used throughout the specification to describe a salt of nitrous acid which is readily soluble in an aqueous system and which readily dissociates into nitrite anion and counterion (generally, metal). Two particularly preferred salts of nitrites for use in the present invention include sodium nitrite and potassium nitrite, although a number of other nitrite salts may also be used in the present invention. The term “nitrite” is used throughout the specification to describe the form in which an amount of a water soluble salt of nitrous acid either in dry or liquid state (preferably, as an aqueous solution) is added to the acid. In general, the nitrite is added to the acid and preferably, both the nitrite and the acid are mixed together in an aqueous solution to which has been added effective amounts of additives such as surfactants, coloring agents, chelating agents and gelling agents, as otherwise described herein. Metal nitrite salts are preferred for use in the present invention.

The term “nitrite ion” is used throughout the specification to describe the nitrite anion of a nitrite salt. In the present application, where the term “nitrite ion” is described in amounts in a given aqueous composition, it is the amount or concentration of the anion which is being referenced, not the amount of total salt concentration which generally contains both a nitrite anion and a metal cation.

The term “acid” is used throughout the specification to describe protic acids, i.e., acids that release hydrogen ions in solution. Acids for use in the present invention include strong inorganic acids such as hydrochloric, sulfuric, and nitric acid; alkylsulfonic acid and benzenesulfonic acid, among other organic sulfonic acids, which, depending upon the end-use of the composition, may be preferably included as dilute acid; organic acids such as citric, fumaric, glycolic, lactic, malic, maleic, tartaric acid, salicylic, citric, propionic, acetic and mandelic, among others, including ethylenediaminetetraacetic acid (EDTA, as the free acid or the monosodium salt), among others; and inorganic acids such as sodium and potassium bisulfate (NaHSO₄ and KHSO₄) and phosphoric acid, among numerous others. It is noted that numerous additional acids may also be used in the present invention. In its broadest aspect, compositions according to the present invention may make use of virtually any acid, to the extent that it provides an initial pH, which when the nitrite-containing part and the acid-containing part are combined produce nitrous acid in amounts effective for the intended purpose. One of ordinary skill will be able to readily determine the type and amount of acid to be used for a particular application.

The term “material inducing an acidic environment therein” is used to describe a material, which, when added to compositions according to the present invention, produces an acidic environmental as a consequence of the interaction of the material with an aqueous solution. Such materials for use in the present invention including for example, carbonic acid, various Lewis Acids, numerous acid inducing metal salts, including, for example, aluminum cations, gadolinium cations, vanadium cations, zirconium cations, zinc cation, more specifically and preferably, for example, aluminum chlorhydroxide, aluminum acetate, aluminum ammonium sulfate, aluminum phenolsulfonate, iron, aluminum, gadolinium and vanadium chlorides, zirconium oxychloride; zinc, cadmium and magnesium salts of chloride, nitrate, sulfate, perchlorate, acetate, citrate, and lactate, among others.

The term “effective amount” is used to describe that amount of a composition, an individual component or a material which is included in compositions according to the present invention in order to produce an intended effect. For example, in the case of an effective amount of an acid, an effective amount is that amount which is included to produce a sufficiently acidic medium to produce nitrous acid in combination with a nitrite salt. An effective amount of nitrite or a nitrite salt is that amount which is effective to produce a desired concentration of nitrous acid after mixing with an appropriate and effective amount of an acid. In the case of a gelling agent, an effective amount of that component is that amount which is effective to gel a final composition (i.e., produce a viscous composition). One of ordinary skill will able to readily determine effective amounts of components or compositions for use to provide an intended effect.

The term “gelling agent” is used throughout the specification to describe a compound or composition which is added to the present compositions in order to increase the viscosity of the composition. Gelling agents which are used in the present invention may be added to the nitrite-containing part (A) or the acid-containing part (B) in amounts effective to gel the solution to which these compounds have been added. Gelling agents for use in the present invention include polysaccharides extracted from legume seeds, such as the galactomannans, including guar gum and locust bean (carob) gum. Other gelling agents include high molecular weight polyoxyalkylene crosslinked acrylic polymers as well as the highly preferred cellulosics such as hydroxymethyl cellulose, hydroxypropyl cellulose, hydroxyethyl cellulose, methyl cellulose, methylpropyl cellulose, among others, including high molecular weight polyethylene glycols, polyacrylamide and polyacrylamide sulfonates, and crosslinked polyvinylpyrrolidones, among others.

As described above, the present invention is directed to a nitrous acid generating composition for disinfecting a substrate. The composition comprises an aqueous solution containing a suitable amount of hydrogen ions derived from either a protic acid or a material which induces an acidic environment therein such as an acid-inducing salt, and a suitable amount of a metal nitrite such as sodium nitrite. Compositions according to the present invention are preferably produced by adding a metal nitrite (either as a dry material or in solution) to an acidic solution. The concentration of hydrogen ion-generating species is such that the amount of nitrite ion in the form of nitrous acid is no more than about 95% by weight of the total nitrite ion in the solution. Preferably, the amount of nitrite in the form of nitrous acid is no more than about 67% by weight of the total nitrite ion concentration in solution.

The percent by weight of nitrite and nitrous acid may be calculated from the ionization constant of nitrous acid and the amount of hydrogen ion in solution produced by partial ionization of the protic acid, or calculated from the pH of a salt-induced acid solution. The hydrogen ion concentration, [H⁺], in a solution of a protic acid, HA, of known molar concentration and whose ionization constant is Kₐ, may be calculated from the following relationship:

$${K}_{a} = \frac{[ {H}^{+} ] [ A ]}{[ HA ]}$$

For solutions where the acidity is induced by the presence of a particular salt, [H⁺] is determined by measurement of the solution's pH and calculation from the negative antilog of that value.

The above relationship may be applied to calculate the relative nitrite and nitrous acid concentrations, where the ionization constant for nitrous acid is 4.5×10⁻⁴. That is:

$$4.5 \times {10}^{- 4} = \frac{[ {H}^{+} ] [ {NO}_{2}^{-} ]}{[ {HNO}_{2} ]}$$

where the hydrogen ion concentration, [H⁺], is the quantity readily determined by ionization of the known amount of the protic acid, HA. This calculation is well known to those skilled in this art.

For the nitrous acid/nitrite system, the following table illustrates representative percentages of both species over a pH range which provides high to low amounts of nitrous acid, as derived from nitrite.

TABLE 1
Percentage of Nitrite as Nitrous Acid
at Varying pH Values

| pH | Nitrous Acid % | Nitrite % |
|---|---|---|
| 1.5 | 98.4 | 1.6 |
| 2.0 | 95.2 | 4.8 |
| 2.3 | 90.9 | 9.1 |
| 2.6 | 83.3 | 16.7 |
| 2.8 | 76.0 | 24.0 |
| 3.0 | 66.7 | 33.3 |
| 3.3 | 50.0 | 50.0 |
| 3.5 | 38.8 | 61.2 |
| 4.0 | 16.6 | 83.4 |
| 4.5 | 6.0 | 94.0 |
| 5.0 | 2.0 | 98.0 |

Aqueous solutions of nitrous acid are unstable, and decompose according to the following equation. Instability increases with increased absolute and relative molar concentrations of the HONO, and with increasing heat:

3HNO₂(aq)⇄H⁺(aq)+2NO(g)+NO₃⁻(aq)+H₂O(I) Equation 1

The reaction is a combination of the two half-reactions, as follows:

2{HNO₂+H⁺+e⁻⇄NO+H₂O} Equation 2

HNO₂+H₂O⇄NO₃⁻3H⁺+2e⁻ Equation 3

Σ=3HNO₂(aq)⇄H⁺(aq)+2NO(g)+NO₃⁻(aq)+H₂O(I)⁻

In addition to the concentration-dependent degradation of nitrous acid, as shown above, nitrous acid will also act as an oxidizing agent in the presence of oxidizable materials, such as microorganisms, according to the first half-cell reaction above (Equation 2), with a redox potential ε⁰=1.00 volts. Accordingly nitrous acid systems are quite destructive of all classes of microorganisms which are susceptible to oxidation, including bacteria, yeasts, molds and viruses. This destruction is well known for other non-specific oxidizing germicides such as bleach (hypochlorous acid), chlorous acid, chlorine dioxide, and iodine. We have discovered that acidified nitrite solutions, upon standing, will generally become either more acidic or less acidic in rough proportion to the pH of the solution, and thus the relative amount of nitrous acid with respect to nitrite ion. At relatively high concentrations of nitrous acid with respect to total nitrite (>˜75%), stored solutions will generally become more acidic. At relatively lower concentrations of nitrous acid, the stored solutions will generally become more alkaline (i.e. less acidic). In one set of experiments the break-point with respect to greater or lesser acid formation occurred at ˜pH 3.7, where the total molar concentration of nitrite [ionic and acid-form] was 0.045M/liter. The data were as follows:

| pH at T = 0 | pH at T = 30 days |
|---|---|
| 2.94 | 2.30 |
| 3.12 | 2.50 |
| 3.35 | 3.25 |
| 3.54 | 3.15 |
| 3.75 | 3.92 |
| 3.90 | 4.35 |

The first solution, at pH 2.94, with a relative nitrous acid level of about 70% (see Table 1), increased in acidity to 2.30, a pH drop of 0.64 units, whereas the last solution, at pH 3.90, and a relative nitrous acid level of about 20%, increased in pH by 0.45 units. The quantity of acid required to reduce the pH in the first solution is, of course, much greater than for the last solution, in large measure because of the logarithmic basis for the pH scale.

Although the direct reason for this difference is not fully understand, the increase or decrease of solution pH is believed to be related to the corresponding contributions of half-cell Equations 2) and 3) above, the former reducing the H⁺ present in solution (i.e. raising the pH) and the latter contributing H+to the medium, and thereby lowering the pH. There may be some involvement of the organic acidifier, which in this experiment was malic acid, in the overall reaction characteristic of the particular combination of nitrite and acid concentrations in this set of solutions. However it is evident from this experiment that it is feasible to adjust the concentrations of nitrite and acid in a preferred composition of this invention such that the solution is stabilized in pH over a prolonged period of time, and capable of being stored as a pre-mixed one-part composition with a commercially-acceptable shelf life.

Nitric Oxide [NO], a paramagnetic species, loses an electron rather easily, to form NO⁺, a reactive species. This reductive tendency is in contrast to the oxidative tendency of chlorine dioxide [ClO₂], another paramagnetic molecule which is a degradation product of chlorous acid. Therein lies a possible reason for the lower corrosion potential of the acidified nitrite system vs. that of acidified sodium chlorite. It is not known, at this point, what aspect(s) of the acidified nitrite system is/are the source of the antimicrobial activity which we have established for this composition, although it appears reasonable that the NO and NO⁺ components play a significant role.

In the acidified NO₂⁻/HONO system, i.e. NO₂⁻+H⁺⇄HONO, where only a fraction of the NO₂⁻ has been converted to the HONO germicidal source, when the latter has been depleted or consumed in solution, additional HONO forms from the residual NO₂⁻. The greater the degree of initial conversion, as a function of the system's pH, the lower the reservoir of NO₂⁻ and the lower the absolute amount of HONO that can subsequently form. But the greater the initial HONO, the greater the potential cidal activity that is available for the system initially. And the greater the potential for the HONO in the system to degrade, inasmuch as stability depends on HONO concentration. Obversely, the lower the initial HONO the greater the reservoir of NO₂⁻, the greater the stability (i.e. prolonged germicidal activity) but the lesser the potential cidal activity. The use of acid activating systems which provide a reservoir of [H⁺]ions, such as α-hydroxy acids, or phosphoric acid, which are not fully ionized initially, allows for additional [H⁺] ions to combine with the NO₂⁻ in the reservoir. Of course, for applications where only initial activity is needed, even mineral acids can serve as the proton source, by selecting their concentrations such that the pH of the system lies in about the 2.5 to 5.0 range. Even such weak acids as propionic acid or carbonic acid can serve as a donor of [H⁺] ions. A nitrite solution charged with gaseous CO₂, particularly under pressure (i.e. carbonic acid), provides a convenient single-solution germicide with extended microbial action. And, for the acid-inducing salt solutions taught by Kross (U.S. Pat. No. 5,820,822) to activate chlorite solutions, the applicability to the nitrite systems is even more appropriate, since less acidic solutions (i.e. higher pH's) are herein required in order to create significant amounts of the nitrous acid as cf chlorous acid. The acid-inducing salts generally cannot achieve the low pH's required for major amounts of the latter to form in their solutions.

In certain embodiments of the invention, the nitrous acid generating composition comprises 0.01 to about 1, typically from about 0.02 to about 0.5, and preferably from about 0.03 to about 0.3 percent by weight of metal nitrite, and a suitable amount of an acid having a pKₐ of from about 2.1 to about 4.8. The pH of this composition is generally less than about 7, typically from about 2.5 to about 7.0.

In certain preferred embodiments of the invention, an acid is used of the formula:

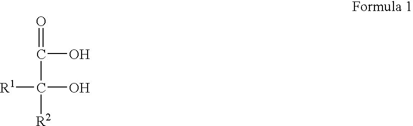

R¹ and R² may be the same or different and may be selected from the group consisting of hydrogen, methyl, —CH₂COOH, —CH₂COO—, —CH₂OH, —CHOHCOOH, —C₆H₅, and —CH₂C₆H₅. The pKₐ of the organic acid may be from about 2.8 to about 4.8.

Other embodiments of the invention may be formulated for a specific disinfecting procedure, or as a result of a specific production method. These embodiments may contain an acid, or acid-inducing component, e.g. carbonic acid or aluminum chloride respectively, which is specifically suited for that procedure or production method. The acid-inducing salts are those taught in U.S. Pat. No. 5,820,822, which is incorporated herein by reference.

While any metal nitrite is useful in the present composition, the alkali and alkaline earth nitrites are preferred because they are readily soluble, readily available and inexpensive. Sodium nitrite, potassium nitrite and ammonium nitrite are preferred. Sodium nitrite is particularly preferred.

The disinfection composition may be used in conjunction with an application medium. The application medium may be any compatible medium including a thickened solution, a gel or a liquid in which water represents a sufficient enough component that the normal equilibrium of the nitrite ion and nitrous acid may exist. An aqueous application medium is preferred. The application medium may contain other additives such as chelating agents (e.g. Na₂H₂ EDTA), surfactants (e.g. alkyl aryl sulfonates such as Nacconol, and nonionic polyoxyalkylene nonylphenols such as Triton N-101), preservatives (e.g. sodium benzoate) or colors (e.g. FD&C Blue #1).

At metal nitrite levels higher than about 0.7%, the concentration of nitrous acid formed upon admixture of a protic acid (or in an acidic aqueous environment otherwise created), in the typical pH range specified, may be in excess of that required for the formation of a metastable nitrous acid solution. These higher concentrations of nitrous acid could promote the formation of nitrous oxide, and nitric oxide therefrom, through the degradation of nitrous acid at too rapid a rate, viz.

3HNO₂⇄H⁺+2NO+NO₃⁻+H₂O; then: 2NO+O₂(air)→2NO₂

However, there are certain applications where rapid disinfection is required, such that a metastable nitrous acid solution is not required, but a high-potency, brief-acting antimicrobial is preferred. In those situations, nitrite levels above 0.7% may be desirable.

Any protic acid, or acidic environment otherwise created, may be used in the present invention so long as the nitrite ion concentration limits described above are met. Suitable protic acids include such inorganic acids as phosphoric acid, and such organic acids as citric, malic, lactic, tartaric, glycolic, mandelic or other structurally similar acids as described in Formula 1 hereinabove. The pKₐ of these organic acids may be generally from about 2.8 to about 4.2, and preferably from about 3.0 to about 4.0. Also suitable are such other acids as salicylic acid, carbonic acid and acetic acid. Inorganic salts, which may be used to induce the requisite pH range, include zinc chloride and aluminum chloride.

The amount of acid, or acid-inducing salt, used in these compositions should be sufficient to lower the pH of the composition to less than about 7, typically from about 2 to about 5, and preferably from about 2.5 to about 4.5. The range of compositions is, of course, very broad, since useful acids range from the very weak, such as carbonic acid with a first pKₐ of 6.37, to the moderately strong, such as tartaric acid with a first pKₐ of 3.03 and phosphoric acid with a first pKₐ of 2.12. Even mineral acids may be used, where sufficiently small amounts are needed to provide the solutions of such Normality that the requisite pH's are achieved.

The nitrous-acid generating compound, i.e. the metal nitrite, is generally kept separate from the acid or acid-generating compound prior to use, in order to avoid premature reaction of the ingredients. Thus, in a general aspect of the present invention, compositions prior to formation of nitrous acid are found in a two part mixture. In general, once the two parts are mixed, there is an initial formation of nitrous acid followed by degradation of the nitrous acid, at a rate dependent on such factors as time, temperature, and concentration of the nitrous acid. The latter will depend upon both the absolute concentration of nitrite ion and the acidity of the system, which determines the degree to which the nitrite ion is converted to nitrous acid, as demonstrated in Table 1 hereinabove. At the upper range of pH values, of about 6.0 to 6.5, the solutions will provide low levels of germicidal activity for several months. At the lower range of pH values, of about 2.5, the high initial cidal capacity of the resulting solution will be significantly reduced within about one day. The pre-mixes may also be combined by in situ application of the individual parts. They may also be applied to various substrates in a manner known to those skilled in this art. They may be sprayed, coated or applied in any other manner depending upon the substrate being treated.

The above-described composition may be used to disinfect various substrates. The term “substrate” as used in the instant specification is intended to cover any type of surface or carrier which could provide a locus for the accumulation of germs (bacteria, yeasts, molds, viruses, -i.e. all types of infectious agents). Obvious examples embrace medical and dental surfaces, including endoscopes, surgical and dental equipment, pharmaceutical and food plants, foods, food containers, human and animal skin and tissues, body fluids and mucous membranes, home areas such as in kitchens, as well as bathroom appliances, food surfaces, sanitation equipment, etc.

Antimicrobial action may be enhanced or extended by inclusion of a variety of agents in either of the pre-mix acid or metal-nitrite compositions, or in the final mixture. These agents may include surface active materials, chelating agents, effervescent compounds and thickeners. These materials must have a minimum tendency to react with the nitrous acid system, or the acidic materials, and be compatible with the other materials in the solutions. The surface active agents, or “surfactants” may be selected from the range of available classes, but non-ionic and anionic surfactants are particularly effective. The amount of surfactant, on the final mix basis, is generally in the range of about 0.001% to about 0.10%, the level depending on the nature and effectiveness of the material in reducing the surface tension of the composition for the desired application. The instant compositions in aerosol form may be effectively used to destroy airborne or atmospheric germs, or may be applied as sprays so as to efficiently cover contaminated surfaces.

Preservatives may also be used in either or both of the pre-mix compositions, to stabilize the solutions. On the basis of the total composition the amount of preservative, from both pre-mix compositions if so present, may generally be from about 0.01 to about 0.08, typically from about 0.01 to about 0.06, and preferably from about 0.02 to about 0.04 percent by weight of the total composition.

When these compositions are used on human or animal skin, they may be typically applied as thickened solutions to facilitate adherence to the skin, and facilitate a greater laydown of germicide. Any thickener which is non-toxic and non-reactive with the nitrous acid system may be used. Many carbohydrate polymers are possible candidates, although some such as the cellulose-based thickeners are less preferred because of their tendency to oxidatively cleave at the β-D-glucose linkage. A preferred thickener is xanthan gum, which is minimally reactive in both the individual pre-mix composition and the final acidified nitrite mix. Other appropriate thickeners include those based on poly(oxyalkylenes) and poly(acrylamides) the latter including the sulfonic acid derivatives thereof, and mineral thickeners such as the silica-based and clay gelling agents.

The amount of thickener or gelling agent which may be used in the thickened, gel composition will vary, depending upon the thickening properties of the gelling agent, the intended application, the level and nature of the acid, the level of the metal nitrite, and other additives employed. Generally, the amount may be from about 0.5 to about 30, typically from about 1 to about 15, and preferably about 1 to about 12 percent by weight of the total composition. Different thickeners may be used in each pre-mix composition (parts A and B), and these levels refer to the combined levels of gelling agent in the total composition.

The amount of metal nitrite in the nitrous-acid generating pre-mix is adjusted so that when the solution (including thickened liquid) is mixed with the acidic component, the specified percentage of metal nitrite will be present in the resulting composition. For example, when two thickened pre-mixes are designed to be mixed in equal parts, which is preferred, the amount of metal nitrite in one part may be generally from about 0.02% to about 2%, typically from about 0.04% to about 1%, and preferably from about 0.06% to 0.6% by weight of that part. Similarly, the amount of acid, or acid-inducing salt in the counterpart pre-mix should be sufficient such that when that pre-mix is combined with the metal nitrite pre-mix, the pH of the resulting composition will be less than about 7, typically from about 2 to about 5, and preferably from about 2.5 to about 4.5. The wide diversity of possible acid sources is such that no particular weight specification for amounts of acid is feasible except on a case-by-case basis, although the acid or material which induces an acid environment is used in the present invention in effective amounts.

The two pre-mix liquids may be combined just prior to application, or may be simultaneously mixed and applied in situ. The compositions of this invention may be applied to various substrates in a manner known to those skilled in this art. The compositions may be sprayed, coated or applied in any manner depending upon the substrate being treated.

The compositions of this invention may be used for skin applications, for example, by applying a small but effective amount of the composition to the affected area of the skin using any means known to those skilled in the art. The composition is allowed to remain on the skin, and evaporate, for a sufficient period of treatment, during which time the loss of water leads to an increased concentration of active agents resulting from the greater resulting acidity. The composition may be reapplied periodically in order to maintain a sustained level of contact of active agents during the course of the treatment. Applications can also be made to mucosal surfaces of an animal, preferably a mammal including a human, to treat infections and inflammatory conditions in a related manner, including such areas as the cheek, the vagina, the peritoneal cavity, and internal sites exposed during surgical procedures.

The compositions may be used to disinfect surfaces, such as in medical and dental operatories and home environments. They are particularly useful in the decontamination of medical equipment, such as endoscopes and hemodialyzers, as well as related liquid pumps and dental water units. The reduced corrosion potential of the acidified nitrite compositions are particularly favored where strong disinfection or sterilization of equipment is needed and where the potential for oxidation would counterindicate the use of oxidants such as chlorous acid systems. The compositions may also be used in personal hygiene formulations, such as oral rinses, toothpastes, soap formulations and douches.

In addition to the unthickened and thickened aqueous solution forms of these compositions, it is also possible to prepare one pre-mix from non-hydrated powders, so that a powdered pre-mix may be combined with an aqueous pre-mix counterpart, such that the concentrations of nitrite and acidic substance in the mixed composition correspond to the specifications of this disclosure. Further, the metal nitrite and the acid, or acid inducing salt, may be provided in solid particulate form in two packages, or in a two-compartment single package wherein the compartments are separated by a suitable seal. One embodiment of such a package uses a water-soluble, heat-sealable, poly(vinyl alcohol) cellulosic film as the packaging materials. Other suitable packaging materials compatible with the composition ingredients are well known to those skilled in the art.

The present invention is illustrated by the following Examples. All parts and percentages in the Examples, as well as the specifications and claims, are by weight, unless otherwise specified. The following examples, which are non-limiting, further describe preferred embodiments within the scope of the present invention. Many variations of these examples are possible without departing from the spirit of the invention.

### EXAMPLE 1

This example illustrates the ability of six acidified nitrite solutions to destroy high levels of the Gram-positive organism Staphylococcus aureus (ATCC 29213), and to a degree consistent with the relative percentage of nitrous acid with respect to total nitrite in the solution. The mixed nitrite/acid solutions, their resulting pH values, and the relative percentages of nitrous acid in the solutions were as shown below. To prepare these solutions, equal parts of a 0.625% NaNO₂ solution and increasing concentrations of malic acid solution were combined as follows:

Characteristics of Acidified Nitrite Solutions

| Sol'n No. | NaNO₂ Premix | Malic Acid Premix | pH of Mix | Total Nitrite as Nitrous Acid |
|---|---|---|---|---|
| 1 | 0.625% | 2.25% | 2.94 | 70% |
| 2 | 0.625% | 1.225% | 3.12 | 60% |
| 3 | 0.625% | 0.812% | 3.35 | 47% |
| 4 | 0.625% | 0.419% | 3.54 | 37% |
| 5 | 0.625% | 0.263% | 3.75 | 28% |
| 6 | 0.625% | 0.156% | 3.90 | 21% |

Procedure: A heavy suspension of the S. aureus was prepared in saline, and 1 part of the suspension was separately combined with 10 parts of each of the above solutions, which had been prepared five minutes before the testing. After five minutes of contact, the mixtures were added to nine volumes of Dey/Engley broth to neutralize the activity and acidity. A 10-fold dilution in saline was made of this mixture. 2 mls of the sample diluted in D/E broth were added to each of five petri plates. 1 ml of the sample diluted in D/E broth was added to each of two petri plates, and 1 ml of the 1/10 dilution of the sample diluted in D/E broth was added to each of two petri plates. Approximately 10 mls of semisolid Trypticase Soy Agar were added to each petri plate, swirled and allowed to harden. The plates were incubated at 35°-37° C. for 48 hours, and the resulting colonies were enumerated. The number of microorganisms in the original suspension was determined by making ten-fold dilutions from 10⁻¹ to 10⁻⁸. Then 1.0 ml portions of the 10⁻⁷ suspension were added to each of two sterile petri plates. 1.0 ml of the 10⁻⁸ suspension was added to each of two sterile petri plates, and 0.1 ml of the 10⁻⁸ suspension was added to each of two sterile petri plates. Approximately 10 mls of semisolid agar were added to each petri plate, swirled and allowed to harden. The plates were incubated at 35°-37° C. for 48 hours, and the resulting colonies were enumerated.

Results

S. aureus Cidal Data*

| Sol'n No. | Recovered cfu | Log Recovery | Log Kill |
|---|---|---|---|
| 1 | 5.4 × 10¹ | 1.7 | 9.1 |
| 2 | 7.0 × 10³ | 3.8 | 7.0 |
| 3 | 4.5 × 10³ | 3.6 | 7.2 |
| 4 | 5.6 × 10⁴ | 4.7 | 6.1 |
| 5 | 6.6 × 10⁵ | 5.8 | 5.0 |
| 6 | >1 × 10⁶ | >6.0 | <4.8 |

*Inoculum suspension contained 7.0 × 10⁸ organisms (i.e. 10.8 logs).

It is obvious that a)- there was significant destruction of the high inoculum of S. aureus in the 5-minute contact period, and b)- the degree of destruction closely parallels the degree of conversion of the nitrite ion to nitrous acid. A 9.1 log kill (>1 billion-fold) was achieved with a solution in which 70% of the nitrite existed in its acidified form of nitrous acid, whereas only 5.0 logs (100,000-fold) were destroyed by the solution with nitrous acid representing 28% of the total nitrite. Even less was destroyed in the 21% nitrous acid (relative) solution.

### EXAMPLE 2

This example illustrates the ability of six acidified nitrite solutions to destroy high levels of the Gram-negative organism Escherichia coli (ATCC 25922). The procedure described in Example 1 was applied in this study as well, using aliquots of the same solutions described in the Table.

The results were as follows:

E. coli Cidal Data*

| Sol'n No. | Recovered cfu | Log Recovery | Log Kill |
|---|---|---|---|
| 1 | 2.7 × 10² | 2.4 | 7.7 |
| 2 | 6.6 × 10⁴ | 4.8 | 5.3 |
| 3 | 9.0 × 10⁰ | 1.0 | 9.1 |
| 4 | 1.4 × 10¹ | 1.1 | 9.0 |
| 5 | 9.9 × 10² | 3.0 | 7.1 |
| 6 | 3.1 × 10³ | 3.5 | 6.6 |

*Inoculum suspension contained 1.2 × 10¹⁰ organisms (i.e., 10.1 logs).

In the case of this Gram-negative organism, the destruction of the inoculum was high in all solutions, apparently independent of pH and thus the relative amount of total nitrite existing as nitrous acid in this series of solutions. It is not known, at this point, whether this difference with respect to the observations in Example 1 is characteristic of the kill mechanism of acidified nitrite solutions with respect to Gram-positive and Gram-negative organisms, or whether it relates to these particular organisms.

### EXAMPLE 3

This example illustrates the ability of six acidified nitrite solutions to destroy high levels of the Gram-negative organism Escherichia coli (ATCC 25922), following 20 days of storage of the mixed solutions at ambient temperatures prior to the testing. The procedure described in Example 1 was applied in this study as well, using aliquots of the same solutions that were evaluated in Examples 1 and 2. The results were as follows:

Results:

The data are presented in the following Table, in which the kills measured on the 20-day old solutions are compared with data obtained on the T=0 mixtures (in brackets).

E. coli Cidal Data on 20-day aged mixtures*

| | | | |
|---|---|---|---|
| Sol'n No. | Recovered cfu | Log Recovery | Log Kill** |

| | | | |
|---|---|---|---|
| 1 | 6.0 × 10¹ | 1.8 | 9.2 [7.7] |
| 2 | 1.5 × 10² | 2.2 | 8.8 [5.3] |
| 3 | 6.0 × 10⁰ | 0.8 | 10.2 [9.1] |
| 4 | >1 × 10⁵ | >5.0 | <6.0 [9.0] |
| 5 | 3.2 × 10⁴ | 3.5 | 7.5 [7.1] |
| 6 | >1 × 10⁶ | >6.0 | <5.0 [6.6] |

*Inoculum suspension contained 9.1 × 10¹⁰ organisms (i.e. 11.0 logs).
**Bracketed data are log kills at T = 0 with the same solutions

About three weeks after preparation, the mixed solutions have retained a significant cidal capacity, as compared with their abilities at T=0. In fact the pH's of these aged solutions, as cf their original values, sheds some light on the greater cidal capacity of the first few solutions tested, viz.

| pH at T = 0 | pH at T = 30 days |
|---|---|
| 2.94 | 2.30 |
| 3.12 | 2.50 |
| 3.35 | 3.25 |
| 3.54 | 3.15 |
| 3.75 | 3.92 |
| 3.90 | 4.35 |

The highest activity, in both fresh and aged solution, appears to occur in the solutions where the pH levels dropped, leading to higher levels of nitrous acid. In these solutions, the nitrous acid and nitrite exist in a ratio of ca. 1:1 and higher. This leads to the speculation that the stability (as well as the activity) of these solutions is related to the presence of a complex ion, such as [HN₂O₄]⁻, analogous to the [Cl₂O₄]⁻ found to exist in ClIO₂ /ClO₂⁻ systems, where the complex [Cl₂O₄]⁻ is conjectured to be an active cidal species, of a higher oxidation potential than ClO₂ alone.

### EXAMPLE 4

This example illustrates the ability of six acidified nitrite solutions to destroy high levels of the yeast Candida albicans (ATCC 10231), and to a degree consistent with the relative percentage of nitrous acid with respect to total nitrite in the solution. The mixed nitrite/acid solutions, their resulting pH values, and the relative percentages of nitrous acid in the solutions were similar to those shown in Example 1.

Procedure: A heavy suspension of the C. albicans was prepared in saline, and 1 part of the suspension was separately combined with 10 parts of each of the above solutions, which had been prepared five minutes before the testing. After five minutes of contact, the mixtures were added to nine volumes of Dey/Engley broth to neutralize the activity and acidity. A 10-fold dilution in saline was made of this mixture. 2 mls of the sample diluted in D/E broth were added to each of five petri plates. 1 ml of the sample diluted in D/E broth was added to each of two petri plates, and 1 ml of the 1/10 dilution of the sample diluted in D/E broth was added to each of two petri plates. Approximately 10 mls of semisolid Sabouraud Dextrose Agar were added to each petri plate, swirled and allowed to harden. The plates were incubated at 20°-25° C. for 72 hours, and the resulting colonies were enumerated.

The number of microorganisms in the original suspension was determined by making ten-fold dilutions from 10⁻¹ to 10⁻⁸. Then 1.0 ml portions of the 10⁻⁷ suspension were added to each of two sterile petri plates. 1.0 ml of the 10⁻⁸ suspension was added to each of two sterile petri plates, and 0.1 ml of the 10⁻⁸ suspension was added to each of two sterile petri plates. Approximately 10 mls of semisolid agar were added to each petri plate, swirled and allowed to harden. The plates were incubated at 20°-25° C. for 72 hours, and the resulting colonies were enumerated.

Results:

C. albicans Cidal Data*

| Sol'n No. | Recovered cfu | Log Recovery | Log Kill | % HONO** |
|---|---|---|---|---|
| 1 | 0 | 0 | >7.86 | 70 |
| 2 | 4 | 0.6 | 7.26 | 60 |
| 3 | 2.4 × 10¹ | 1.38 | 6.48 | 47 |
| 4 | 2.1 × 10⁴ | 4.32 | 3.54 | 37 |
| 5 | >1 × 10⁶ | >6 | <˜1 | 28 |
| 6 | >1 × 10⁶ | >6 | <˜1 | 21 |

*Inoculum suspension contained 7.3 × 10⁷ organisms (i.e. 7.86 logs).
**% of total nitrite ion present as nitrous acid

The destruction of the C. albicans yeast is quite significant, particularly for the solutions below about 3.5, where the nitrous acid is present in a ratio of about 1:1 with respect to ionic nitrite (i.e. above about 50% of total nitrite as HONO). Thereafter the fall off in kill is rather dramatic, at higher pHs. For this organism, as for the S. aureus of Example 1, this suggests that a 1:1 adduct of nitrous acid and nitrite may be providing particularly effective cidal capacity in this system.

### EXAMPLE 5

This example illustrates the ability of six acidified nitrite solutions to destroy high levels of the mold Aspergillus niger (ATCC 6275). The mixed nitrite/acid solutions, their resulting pH values, and the relative percentages of nitrous acid in the solutions were similar to those shown in Example 1, and the procedure followed paralleled that provided in Example 4.

Results: A. niger Cidal Data*

| Sol'n No. | Recovered cfu | Log Recovery | Log Kill |
|---|---|---|---|
| 1 | 18 | 1.26 | 7.14 |
| 2 | 83 | 1.92 | 6.48 |
| 3 | 30 | 1.48 | 6.92 |
| 4 | 37 | 1.57 | 6.83 |
| 5 | 0 | 0 | >8.40 |
| 6 | 0 | 0 | >8.40 |

*Inoculum suspension contained 2.5 × 10⁸ organisms (i.e. 8.40 logs).

In this study, the A. niger mold was effectively destroyed by all solutions, seemingly even more so by the solutions where the nitrous acid species represented a smaller fraction of total nitrite than was seen for the more effective ones Examples 1 and 5. The reason for these disparities has yet to be elucidated.

### EXAMPLE 6

This example illustrates the inherent compatibility of acidified nitrite solutions with red meat and poultry tissue, specifically in their minimum impact on the color of the animal tissue following contact with the disinfecting solution. The example includes a comparison with a representative acidified chlorite disinfecting solution, and with a water control.

Procedure: A disinfecting solution was prepared by combining equal parts of the following solutions: 0.625% Sodium Nitrite; 0.812% Malic Acid. The pH of the mixed solution was 3.35, and the calculated molar amount of nitrous acid was 47% with respect to total nitrite ion. Another solution was prepared by combining equal parts of the following solutions: 0.50% Sodium Chlorite; 3.0% Lactic Acid. The pH of the mixed solution was 2.77, and the calculated molar amount of chlorous acid was 13% with respect to total chlorite ion. Within two minutes of their preparation, small pieces of fresh pork loin were immersed in each of the solutions for 15 seconds, and then removed. After another 45 seconds, the surfaces were rinsed with tap water, and the two pieces of meat were compared in color, and with a similar piece that had been exposed only to tap water. The exposure time employed is considered to be more than adequate for decontamination of animal tissue during processing. The results of the comparison are as follows:

| | | |
|---|---|---|
| Immersion Liquid | Observation | . |

| | |
|---|---|
| Nitrous Acid | No perceptible change in color or texture |
| Chlorous Acid | Color of meat changed to tan / light brown |
| Water | No perceptible change in color or texture |

This Example demonstrates that a nitrous acid solution, at a concentration which has been shown to be an effective germicide, can be used to disinfect animal tissue with no impact on the organoleptic properties of the meat. In contrast a chlorous acid solution, representative of a commercially-available disinfecting solution (LD®, sold by Alcide Corporation) can significantly affect the color of the animal tissue in a negative manner.

### EXAMPLE 7

This example illustrates the compatibility of an acidified nitrite disinfecting solution with metals susceptible to oxidation, as compared with the effect of an acidified chlorite system. Many medical and dental instruments contain such oxidizable metals, and related materials, and cannot be safely disinfected or sterilized with chlorous acid antimicrobial formulations.

Procedure: Nitrous acid and chlorous acid solutions of similar compositions to those described in Example 4 were used in this study. Individual disposable razor blades, presumed to be comprised of a “400-series” stainless steel alloy, were suspended by thin threads into one of the two described solutions for one day, at ambient temperatures. A blade sample was also immersed in tap water for that same period. The exposure time employed is considered to be more than adequate for decontamination of candidate equipment. The results of the comparison are as follows:

| | | |
|---|---|---|
| Immersion Liquid | Observation | . |

| | |
|---|---|
| Nitrous Acid | No perceptible change in appearance |
| Chlorous Acid | Blade edge had rust spots along the edge |
| Water | No perceptible change in appearance |

This Example demonstrates that a nitrous acid solution, at a concentration which has been shown to be an effective germicide, can be used to disinfect or sterilize metal-containing equipment or instruments, with no impact on the integrity of the equipment or instrument. In contrast a chlorous acid solution, representative of a commercially-available disinfecting solution (LD®, sold by Alcide Corporation) can significantly affect the integrity of the metal substrate.

It is clear that the present invention is well adapted to carry out the objects, and achieve the ends and advantages mentioned at the outset. While currently preferred embodiments of the invention have been described for purposes of this disclosure, numerous modifications may be made which will readily suggest themselves to those skilled in the art, and which are encompassed within the spirit of he invention disclosed, and as defined in the appended claims.

### Other References

Friedman, H. L. “On the Ultraviolet Absorption Spectra of Uninegative Ions”, J. Chem. Physics, (1953), Vol. 21, No. 1, p. 319 et seq.

Masschelein, W J; (1979) Chlorine Dioxide; Chemistry and Environment Impact of Oxychloride Compounds. Ann Arbor Science, Mich.

We claim:

1. A process for disinfecting a substrate comprising contacting said substrate with an aqueous composition consisting essentially of water and an effective amount of a protic acid, or a material inducing an acidic environment therein, and an effective amount of a water soluble nitrite salt to produce nitrous acid from said acid and said nitrite salt, said composition containing an amount of nitrous acid which is no more than about 95% by weight of the total amount of nitrite ion and nitrite as nitrous acid in said composition.

2. The process of claim 1 wherein the nitrite salt is present at a level of from about 0.01% to about 1.0% by weight based on the total weight of the composition.

3. The process of claim 1 wherein the nitrite salt is sodium nitrite.

4. The process of claim 1 wherein the protic acid is an organic acid.

5. The process of claim 4 wherein the organic acid is selected from a group with pKₐ values in the range of about 2.8 to about 4.8.

6. The process of claim 5 wherein said acid is selected from the group consisting of citric acid, lactic acid, malic acid, tartaric acid, glycolic acid, mandelic acid, salicylic acid and mixtures thereof.

7. The process of claim 5 wherein the organic acid ranges from about 0.03% to about 3% by weight of the total composition.

8. The process of claim 1 wherein the protic acid is an inorganic acid.

9. The process of claim 8 wherein the inorganic acid is selected from the group containing nitric acid, hydrochloric acid, sulfuric acid, sodium hydrogen sulfate, phosphoric acid, and carbonic acid.

10. The process of claim 1 wherein the material inducing an acidic environment is an inorganic salt which creates a pH in said aqueous composition of less than about 7.

11. The process of claim 9 wherein the material inducing an acidic environment is selected from the group consisting of the metal cations selected from the group consisting of iron, aluminum, gadolinium, vanadium, zirconium, zinc, and mixtures thereof, in combination with anions effective for rendering said metal salt electrically neutral and water soluble.

12. The process of claim 1 wherein the substrate is selected from groups consisting of skin, tissue, body fluids and mucous membranes.

13. The process of claim 1 wherein said substrate is a metal substrate.

14. A nitrous acid-generating composition for use in disinfecting a substrate comprising an aqueous solution consisting essentially of an effective amount of a protic acid, or a material inducing an acidic environment therein, and an effective amount of a nitrite salt to produce nitrous acid, said nitrous acid comprising no more than about 95% by weight of the total amount of nitrite ion and nitrite as nitrous acid in said composition.

15. The composition of claim 14 wherein said nitrite salt ranges from about 0.01% to about 1.0% by weight of the total weight of said composition.

16. The composition of claim 14 wherein the nitrite salt is sodium nitrite.

17. The composition of claim 14 wherein the protic acid is an organic acid.

18. The composition of claim 17 wherein the organic acid has a pKₐ value in the range of about 2.8 to about 4.8.

19. The composition of claim 16, wherein said acid is selected from the group consisting of citric acid, lactic acid, malic acid, tartaric acid, glycolic acid, mandelic acid, salicylic acid, and mixtures thereof.

20. The composition of claim 16 wherein said organic acid ranges from about 0.03% to about 3% by weight of the total composition.

21. The composition of claim 14 wherein the protic acid is an inorganic acid.

22. The composition of claim 21 wherein the inorganic acid is selected from the group containing nitric acid, hydrochloric acid, sulfuric acid, sodium hydrogen sulfate, phosphoric acid, and carbonic acid.

23. The composition of claim 14 wherein the material inducing an acidic environment is an inorganic salt which creates a pH in the disinfecting solution of less than about 7.

24. The composition of claim 23 wherein the material inducing an acidic environment is a cation selected from the group consisting of iron, aluminum, gadolinium, vanadium, zirconium, zinc, and mixtures thereof, in combination with anions effective for rendering said metal salt electrically neutral and water soluble.

25. The composition of claim 14 wherein the substrate is selected from skin, tissue, body fluids and mucous membranes.

26. The composition according to claim 14 further comprising effective amounts of at least one additional additive selected from the group consisting of surfactants, chelating agents, coloring agents and gelling agents.
